(19) United States
(12) Patent Application Publication
(10) Pub. No.: US 2026/0158224 A1
(43) Pub. Date: Jun. 11, 2026

(54) Smart Device Assisted Syringe Filling and Dosing System

(71) Applicant: R S Brain, LLC, Hudson, NH (US)

(72) Inventors: Joseph Geringer, Hudson, NH (US); Robert Carpino, Nashua, NH (US)

(73) Assignee: R S Brain, LLC, Hudson, NH (US)

(21) Appl. No.: 18/977,382

(22) Filed: Dec. 11, 2024

(51) Int. Cl. A61M 5/315; A61M 5/31

(52) U.S. Cl. CPC A61M 5/31546; A61M 5/31578; A61M 2005/3125

(57) ABSTRACT

A system for accurately dosing medication using smart devices and injection devices. The system includes a smart device configured to provide visual aids and feedback mechanisms to help users verify the correct dosage. The smart device displays dosage information in a clear and understandable manner, reducing the likelihood of errors. The system also provides real-time feedback and guidance, ensuring users can confidently manage their medication. The integration of smart technology into the dosing system enhances accuracy and ease of use, offering a more reliable and user-friendly approach to medication management.

### FIELD OF THE DISCLOSURE

The present disclosure relates to medical devices, specifically to systems and methods for accurately dosing medication using smart devices and injection devices.

### BACKGROUND

Accurate dosing of medication is important in the treatment of diseases. Ensuring that patients receive the correct amount of medication can significantly impact the effectiveness of the treatment and the patient's overall health. Traditional methods of dosing often rely on manual processes, which can introduce errors. These errors often result in administering too little or too much medication, potentially leading to adverse health effects. Therefore, there is a need for systems that provide clear, precise, and intuitive methods for managing medication dosage.

Existing solutions for medication dosing often lack the precision and intuitive interfaces needed to ensure users can confidently manage their medication. For example, manual dosing methods can be prone to human error, and even small inaccuracies can have significant consequences.

One present solution to this issue are automatic systems that focus on determining dosage based on flow rate, incorporating time clocks and memory devices to store sensor data and corresponding time stamps. However, these systems do not provide a comprehensive solution for visualizing and verifying dosage amounts and are quite expensive to produce and distribute.

Other systems take into account user input regarding a disease, ensuring that the user satisfies criteria defining a combination of events related to managing the disease. These systems focus on detecting if the input satisfies the criteria, ensuring a target dosage is optimal, but still do not provide a way for a user to visualize and verify actual dosage amounts.

There is thus a need for a cost-effective dosing system that addresses the shortcomings of existing solutions by providing a clear, precise, and intuitive method for managing and confirming medication dosage. Such a system should incorporate smart technology to enhance accuracy and ease of use, offering a more reliable and user-friendly approach to medication management. This system should provide visual aids and feedback mechanisms to help users verify the correct dosage, ensuring that patients receive the appropriate amount of medication for their treatment and increasing their confidence.

### SUMMARY

The present disclosure provides a system that addresses patient needs as well as the shortcomings of existing solutions by offering a clear, precise, and intuitive system and method for managing medication dosage. Embodiments incorporate a user device, such as a smartphone, into the syringe filling process, in embodiments using an adapter to hold a syringe and vial in place adjacent a user interface, such as a display, of the user device during filling.

In embodiments, the user device may be configured, such as through installation and use of an application, to display dosage information in a clear and understandable manner, reducing the likelihood of errors. The system may also offer real-time feedback and guidance during syringe filling or even automatically fill the syringe from the vial in response to user commands entered through the application. Notably, embodiments provide imagery on the display that is aligned with the syringe and adapter and against which the filled syringe and adapter may be compared to confirm correct dosage, ensuring that users can confidently manage their medication.

This integration of smart technology that is likely already owned by the user into the dosing system represents a significant advancement in patient care, offering a more reliable and user-friendly approach to medication management while enhancing accuracy and minimizing the costs associated therewith.

One embodiment of the present disclosure provides a system for accurately dosing medication, the system comprising: a syringe filling adapter configured to hold a syringe and vial such that the syringe is inserted into the vial, the syringe filling adapter further comprising a plunger handle configured for connection to a plunger of the syringe and axial translation, relative to the syringe.

Another embodiment of the present disclosure provides such a system, further comprising a stop associated with the plunger handle and configured to adjustably restrict motion of the plunger handle.

A further embodiment of the present disclosure provides such a system, further comprising a sensor PCB attached to the syringe for capacitive sensing of the plunger position.

Yet another embodiment of the present disclosure provides such a system, further comprising an actuator linked to the plunger handle, the actuator configured to advance or retract the plunger of the syringe in response to commands from a device.

A yet further embodiment of the present disclosure provides such a system, wherein the actuator is a muscle-wire actuator.

Still another embodiment of the present disclosure provides such a system, wherein the actuator comprises a motor configured to drive a worm-gear.

A still further embodiment of the present disclosure provides such a system, wherein the device is configured to provide an input interface for commanding the syringe filling adapter to fill a syringe disposed therein to a target dosage.

Even another embodiment of the present disclosure provides such a system, wherein the syringe filling adapter is configured to mount to a device comprising a touch screen display and to hold the syringe adjacent the display and parallel to an edge of the display, and wherein the display is configured to display a target dosage graphic that provides at least one reference point to which the plunger handle should be aligned to fill the syringe to a target dosage.

An even further embodiment of the present disclosure provides such a system, wherein the target dosage graphic comprises a numerical value corresponding to the target dosage.

A still even another embodiment of the present disclosure provides such a system, wherein the target dosage graphic comprises a 1:1 scale graphical representation of the syringe filling adapter with plunger handle extended to a position corresponding to the target dosage, such that the syringe filling adapter completely covers the target dosage graphic when the syringe is filled to the correct dosage.

A still even further embodiment of the present disclosure provides such a system, wherein the target dosage graphic comprises a graphic to which the plunger handle should abut to fill the syringe to the target dosage.

Still yet another embodiment of the present disclosure provides such a system, wherein the display of the device is a capacitive, touch-sensitive display and wherein the syringe filling adapter includes a capacitive touch member configured to move with the plunger handle, adjacent the display, and thereby to inform the device of a current plunger position.

A still yet further embodiment of the present disclosure provides such a system, wherein the device is configured to obtain an initial plunger handle position through recording the location of the capacitive touch member and then to use an offset from this position to generate the target dosage graphic.

Even yet another embodiment of the present disclosure provides such a system, wherein the device is configured to provide visual, tactile, and/or audible feedback to help users verify the target dosage.

An even yet further embodiment of the present disclosure provides such a system, wherein the feedback is provided in response to the pulling of the plunger handle to the target dosage.

Still even yet another embodiment of the present disclosure provides such a system, wherein the feedback is provided as the plunger handle approaches the target dosage.

One embodiment of the present disclosure provides a system for accurately dosing medication, the system comprising: a display; at least one processor; and one or more memories coupled to the at least one processor and having programming that causes the system to perform operations including: accepting a target dosage from a user; generating a target dosage graphic that corresponds to a syringe or syringe filling adapter with syringe filled to the target dosage; and displaying the target dosage graphic on the display.

Another embodiment of the present disclosure provides such a system, wherein the target dosage graphic comprises a graphic to which a plunger handle of the syringe filling adapter should abut to fill a syringe held in the syringe filling adapter to the target dosage.

A further embodiment of the present disclosure provides such a system, wherein the target dosage graphic comprises a 1:1 scale graphic of a syringe filled to the target dosage.

One embodiment of the present disclosure provides a system for accurately dosing medication, the system comprising: a syringe filling adapter configured to hold a syringe and vial such that the syringe is inserted into the vial, the syringe filling adapter further comprising a plunger handle configured for connection to a plunger of the syringe and axial translation, relative to the syringe; a stop associated with the plunger handle and configured to adjustably restrict motion of the plunger handle; wherein the syringe filling adapter is configured to mount to a device comprising a touch screen display and to hold the syringe adjacent the display and parallel to an edge of the display, wherein the display is configured to display a target dosage graphic that provides at least one reference point to which the plunger handle should be aligned to fill the syringe to a target dosage, wherein the display of the device is a capacitive, touch-sensitive display, wherein the syringe filling adapter includes a capacitive touch member configured to move with the plunger handle, adjacent the display, and thereby to inform the device of a current plunger position, and wherein the device or syringe filling adapter is configured to provide visual, tactile, and/or audible feedback to help users verify the target dosage.

The features and advantages described herein are not all-inclusive and; many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a perspective view showing a syringe filling adapter mounted to a user device, in accordance with embodiments of the present disclosure;

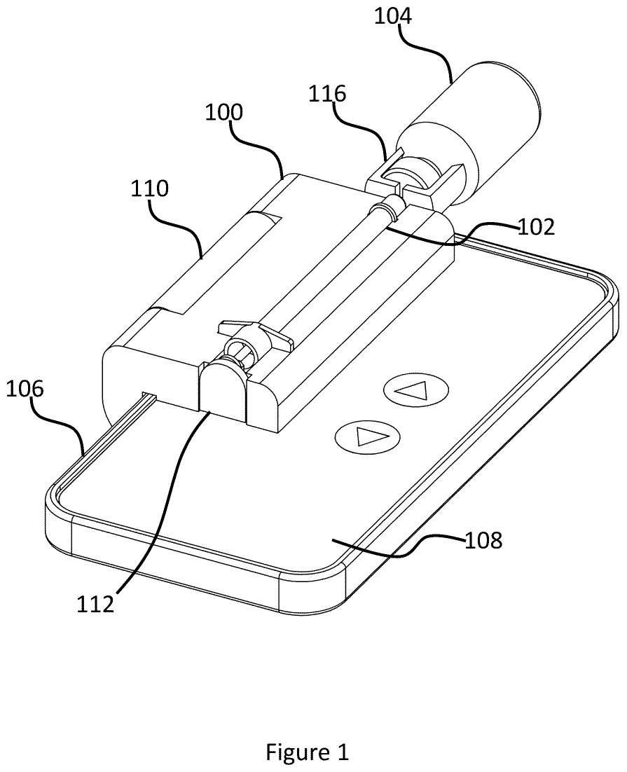

FIG. 2A shows a User Interface (UI) of the user device and syringe filling adapter of FIG. 1, the user interface displaying a selected dose in units and providing pressable arrows for adjusting the dose, in accordance with embodiments of the present disclosure;

FIG. 2B shows the user interface of FIG. 2A where a larger dose is selected, with a graphic depicting the location of a plunger handle associated with the adapter and connected to a plunger of a syringe disposed in the adapter when the syringe is filled to the selected dose, which may be depicted on the UI, providing a user a means to confirm the selected dosage, in accordance with embodiments of the present disclosure;

FIG. 2C shows a cross-sectional view of the syringe filling adapter depicted in FIG. 2A, along section line C-C, showing how the adapter mounts to the user device, in accordance with embodiments of the present disclosure;

FIG. 2D shows a cross-sectional view of the adapter depicted in FIG. 2A, along section line A-A, showing how the adapter interacts with the UI of the user device, in accordance with embodiments of the present disclosure;

FIG. 2E shows a detailed view of a section B of FIG. 2D, better showing a conductive rubber cap affixed to a portion of the plunger handle of the adapter and contacting the UI, providing a means by which plunger position and movement can be established, in accordance with embodiments of the present disclosure;

FIG. 3 provides a perspective view of a motor-driven worm gear mechanism that is configured to drive a stop that limits motion of the plunger handle of the adapter, the mechanism being contained within embodiments of the adapter, in accordance with embodiments of the present disclosure;

FIG. 4 illustrates the stop of FIG. 3 in an intermediate position and shows how motion of the plunger handle, and thus a plunger of a syringe disposed in the adapter, is limited by the location of the stop, in accordance with embodiments of the present disclosure;

FIG. 5 provides a perspective view of version of the worm gear mechanism first shown in FIG. 3 that relies on a knob to adjust the position of the stop, in accordance with embodiments of the present disclosure;

FIG. 6 shows a perspective view of a standalone syringe filling apparatus configured to hold a syringe and vial, in accordance with embodiments of the present disclosure;

FIG. 7A shows a front elevation view of the standalone syringe filling apparatus, in accordance with embodiments of the present disclosure;

FIG. 7B shows a cross-sectional view of the standalone syringe filling apparatus along section line C-C.

FIG. 8 shows a rear elevation view of the standalone syringe filling apparatus, showing control buttons, in accordance with embodiments of the present disclosure;

FIG. 9A illustrates a muscle-wire actuator disposed within the standalone syringe filling apparatus, in accordance with embodiments of the present disclosure;

FIG. 9B shows the muscle-wire actuated dosing mechanism of FIG. 9A, better showing details of the ratchet and pall, in accordance with embodiments of the present disclosure;

FIG. 10 shows a user device displaying a numerical dosage value and a 1:1 graphical representation of a syringe filled to the correct dosage.

### DETAILED DESCRIPTION

In treating serious diseases, especially those of a chronic nature, patients are often required to administer their own injections using a syringe 102. Accurate dosing of injectable medication is important in the treatment of such diseases and often difficult for patients, who may also be suffering from poor eyesight and/or dexterity. It is, however, of utmost importance that patients receive the correct amount of medication, since this can significantly impact the overall effectiveness of the treatment and the patient's overall health.

Traditional methods of dosing, which most often involve filling a syringe 102 from a vial 104 of medication manually, are cost-effective, but can introduce errors that result in administering too little or too much medication, potentially leading to adverse health effects. Existing solutions for automatic medication dosing, however, are often expensive and out of reach of a typical patient due to the incorporation of single-purpose disposable components, such as cartridges of medication and needles, sensitive electronic components, such as flow meters, and their design, overall, being somewhat complex.

The present disclosure provides a system and method that addresses the shortcomings of existing solutions by offering a clear, precise, and intuitive method for managing medication dosage. Embodiments incorporate a user device 106, such as a smartphone, into the filling process, in embodiments using a syringe filling adapter 100 to hold a standard syringe 102 and vial 104 in place against a user interface 108, or display, of a user device 106 during filling.

In embodiments, the user device 106 may be configured, such as through installation and use of a software application, to display dosage information in a clear and understandable manner on a user interface 108 of the user device 106, reducing the likelihood of errors. The system may also offer real-time feedback and guidance during syringe 102 filling or even automatically fill a syringe 102 after receiving a command from the user device 106. Embodiments may further provide reference imagery against which a filled syringe 102 may be compared to confirm correct dosage, ensuring that users can confidently manage their medication. By either including features that allow the position and orientation of the syringe filling adapter 100 to be determined by the user device 106 or fixed to a repeatable position thereon, the reference imagery can be aligned to a plunger handle 112 associated with the syringe filling adapter 100 that is, in embodiments, fixed to the plunger of the syringe 102. This arrangement allows users to directly compare the syringe's plunger position with a displayed target dosage graphic, providing a redundant check to ensure the correct dosage is administered.

This integration of smart technology that is likely already owned by the user into the dosing system represents a significant advancement in patient care, offering a more reliable, user-friendly, and cost-effective approach to medication management while enhancing accuracy.

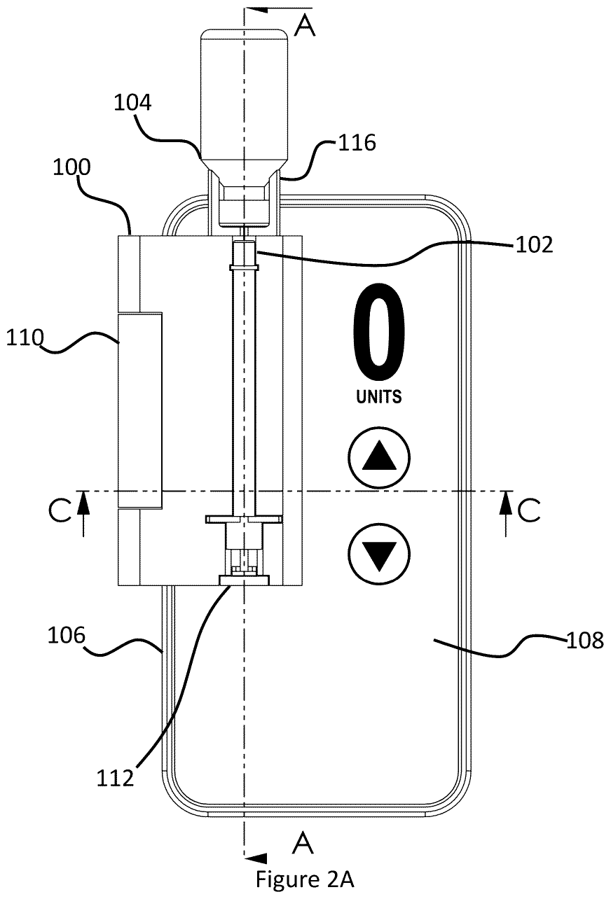

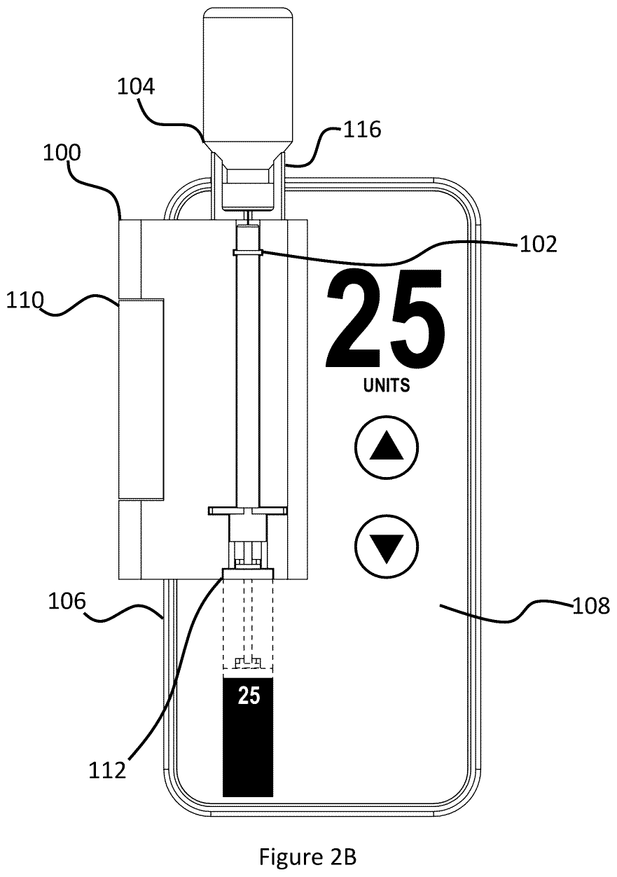

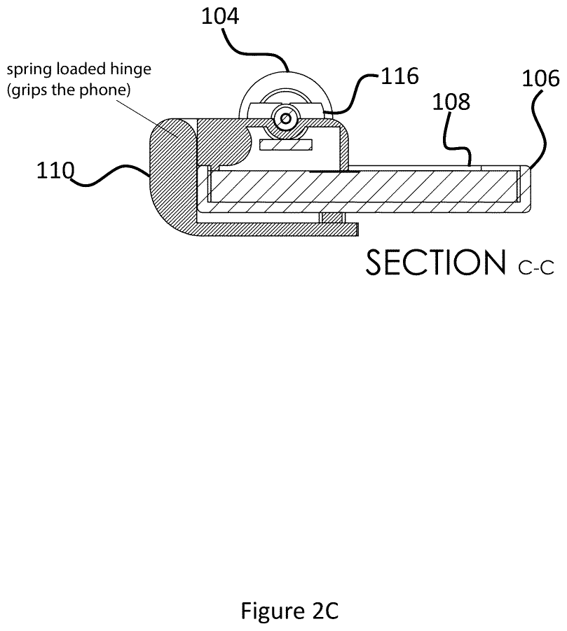

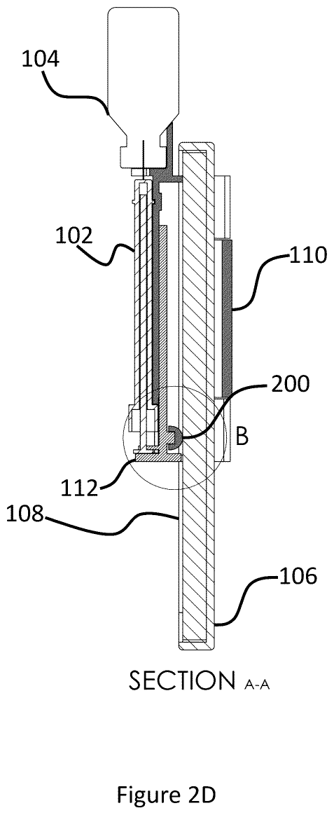

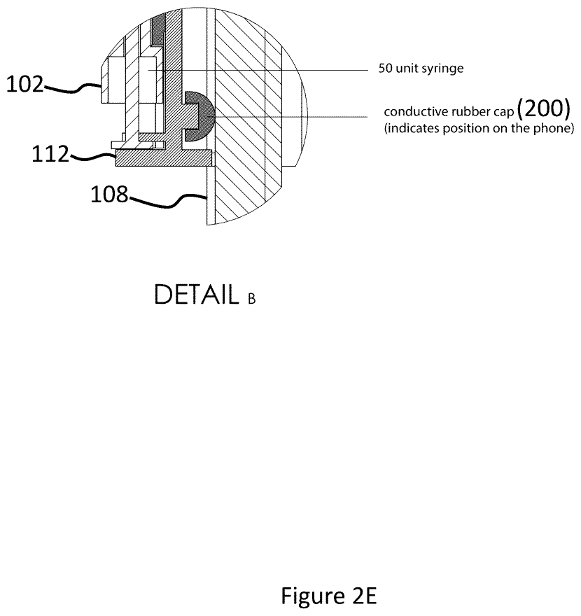

FIGS. 2A through 2E provide various views showing a syringe filling adapter 100 in accordance with the teachings of the present disclosure mounted to a user device 106. More specifically, FIG. 2A shows the syringe filling adapter 100 holding an empty syringe 102 and vial 104 (using a vial adapter 116) containing medication. The syringe filling adapter 100 is held adjacent a user interface 108 of the user device 106 using a hinge 110 and the syringe 102 itself is held parallel to a plane defined by the user interface 108; in embodiments the syringe 102 is also held parallel to an edge of the user interface 108. In further embodiments, the syringe filling adapter 100 may locate itself using a corner or other feature of the user device 106.

In FIG. 2A, the selected amount of medication is 0 units and the plunger handle 112, which may be fixed to the plunger of the syringe 102 such that the two move together as one, is fully depressed.

In embodiments, the arrows displayed on the user interface 108 are used to adjust a selected dose, with units being replaced by the appropriate unit of measure for the dose being measured. In other embodiments, the selected dose is calculated automatically, based on user inputs, predetermined, or set remotely by a physician or similar.

In embodiments, the vial adapter 112 is readily removable from the syringe filling adapter 100, allowing it to be replaced with a vial adapter 116 of a different size should the user wish to use the syringe filling adapter 112 with a vial 104 of a different size. Likewise, the area in which the syringe 102 sits may feature replaceable or adjustable components, allowing the use of syringes 104 of different sizes.

FIG. 2B shows the system of FIG. 2A where 25 units of medication is selected. The plunger handle 112 extended to the selected dose of medication is drawn in broken lines. Below the extended plunger handle 112 is a graphic against which the plunger handle 112 must abut to establish an appropriate dose of medication, allowing a user to establish an initial measurement using gradations on the syringe itself OR by abutting the plunger handle 112 to the aforementioned graphic. Once a dose is measured, the user can then confirm using the alternative method of measuring a selected dose. The plunger handle 112 may be configured for manual movement by a user or may be driven, such as by a motor, solenoid, muscle wire (e.g. shape memory alloy, such as Nitinol), or the like.

By displaying a target dosage graphic that the plunger handle 112 abuts when the syringe 102 is filled to the correct dosage, the system provides a straightforward and reliable way for users to verify dosage without requiring complex calculations or measurements. This feature simplifies the dosing process and minimizes the risk of human error, making it easier for patients to manage their medication accurately.

In embodiments, such as that depicted in FIG. 2B, the selected dose and/or other information may be displayed within the graphic.

In embodiments, a 1:1 scale graphic depicting the syringe 102 disposed in the syringe filling adapter 100 and filled to the selected dosage is used to establish or confirm measurements, with a user aligning the plunger handle to the 1:1 scale graphic to establish or confirm a selected dosage. In embodiments, the scale graphic may be used by a user to locate the syringe filling adapter 100 on the user interface 108.

Now referring to FIG. 2C, details of how the syringe filling adapter 100 attaches to a user device 106 are shown. In the depicted embodiment, a hinge 110, which may be spring loaded, is used to attached the syringe filling adapter 100 to the user device 106. Alternative methods of attachment, however, such as incorporating the syringe filling adapter 100 into a model-specific case or using a set screw or similar arrangement, would be apparent to one of ordinary skill in the art.

FIGS. 2D and 2E illustrate a feature of the syringe filling adapter 100 by which the user device 106 can measure the current position of the plunger handle 112 as well as any changes thereto. Such a feature allows the user device 106 to provide real-time feedback and guidance during the syringe filling process, enhancing user confidence and accuracy. For instance, the user device 106 may provide visual, tactile, and audible feedback to help the user verify the correct dosage, with the feedback being triggered by the movement of the plunger to the correct position, for example by measuring a first position of the plunger handle 112 and then monitoring the location of the plunger handle 112 and providing the feedback when the plunger handle 112 reaches a distance from the starting point that corresponds to the selected dose, which may also be referred to herein as the target dose. This distance may be calculated, for example, based on the internal diameter of the syringe 102. Alternatively or additionally, feedback may be provided as the plunger handle 112 approaches the correct position, with the feedback intensifying as the plunger handle 112 nears the correct position.

This may be accomplished by including a feature on an underside of the plunger handle 112 and/or syringe filling adapter 100 that is intended to interact with the user interface 108 of the user device 106.

The user device 106 may be configured to obtain an initial plunger handle position through recording the location of the capacitive touch member and then to use an offset from this position to generate at least one reference point associated with the target dosage graphic. In embodiments, the user device 106 may be further configured to instruct a user to conduct a calibration procedure, which may involve extending and retracting the plunger handle 112 such that the user device can measure the range of extension of the plunger handle 112, allowing the precise position of the syringe filling adapter 100 to be ascertained.

Where the user interface 108 is a capacitive, touchscreen display, this feature could be a conductive rubber cap 200 affixed to a bottom of the plunger handle 112 such that it is in light contact with the user interface 108; software installed on the user device 106 of such embodiments may be configured to ignore such inputs in a given region, to allow user interaction with the user interface 108. User inputs may be blocked during filling of the syringe 102 to allow the user device to read a current plunger handle 112 location. These embodiments are merely exemplary, however, as a person of ordinary skill in the art would be aware that there are many other ways that this functionality could be accomplished.

In another embodiment, the syringe filling adapter 100 incorporates a sensor PCB configured to sense a position of the plunger or plunger handle 112 (e.g. capacitive sensing, use of an encoder wheel, or similar), allowing the user device 106 to detect and display the exact dosage being drawn.

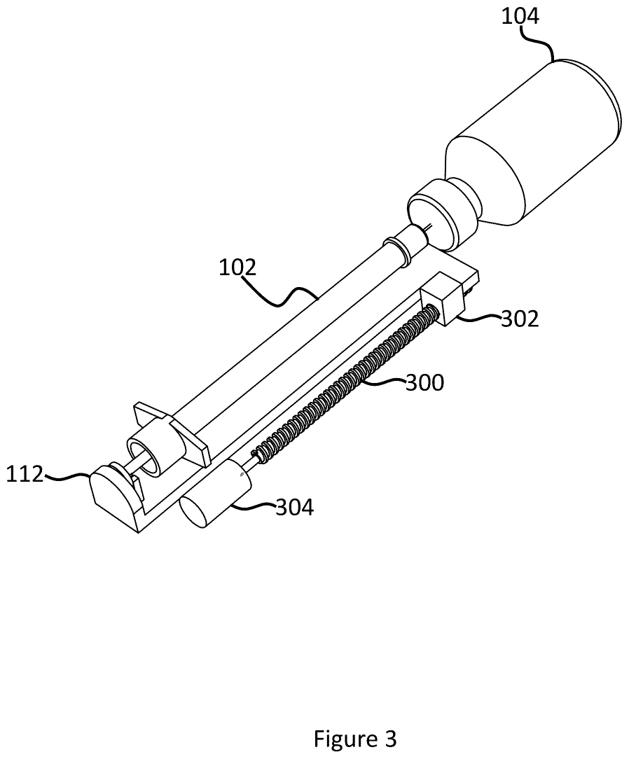

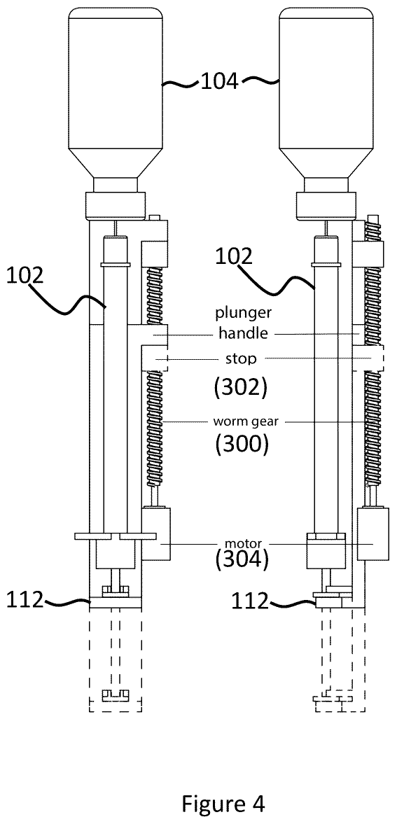

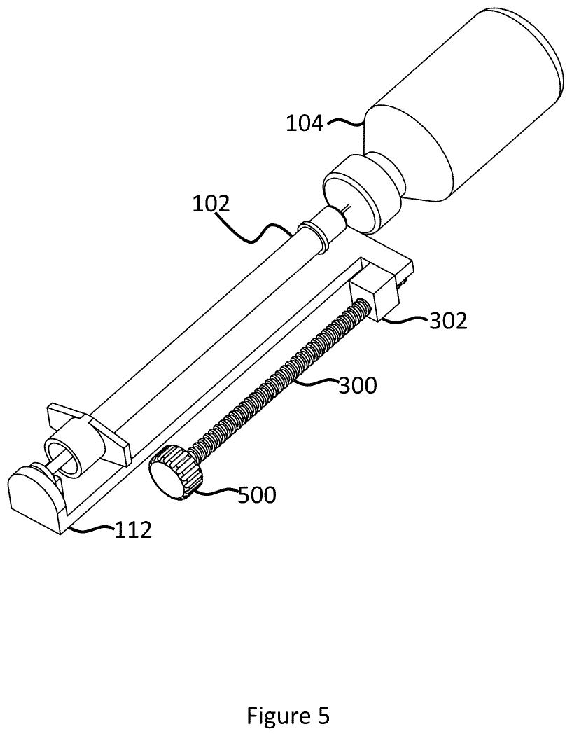

FIGS. 3-5 show embodiments of the plunger handle 112 that include a positive, adjustable stop 302 configured to limit plunger handle 112 travel and thereby ensure that the maximum does is limited to the target dose while making it easier for the user to determine the appropriate plunger handle 112 position needed to deliver the full, intended dose. This may be accomplished using a threaded rod 300 that is rotatably disposed on a portion of the plunger handle 112 distal from the portion that engages with the plunger of the syringe 102. The stop 302 is threaded onto the threaded rod 300 and restricted from rotation by the body of the syringe filling adapter 100 (not shown), the plunger handle 112 itself, or similar, such that rotation of the threaded rod 300 results in an axial translation of the stop 302 along the threaded rod 300. Rotation of the threaded rod 300 may be accomplished manually, such as through the use of a knob 500 disposed on a proximal end of the threaded rod 300 (see, for example, FIG. 5), or automatically, such as through the use of a motor 304 disposed on the proximal end of the threaded rod 300 (see, for example, FIGS. 3 and 4).

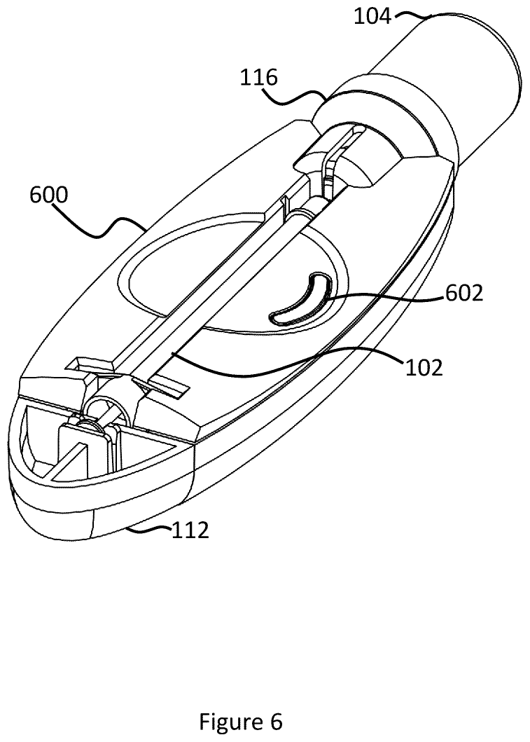

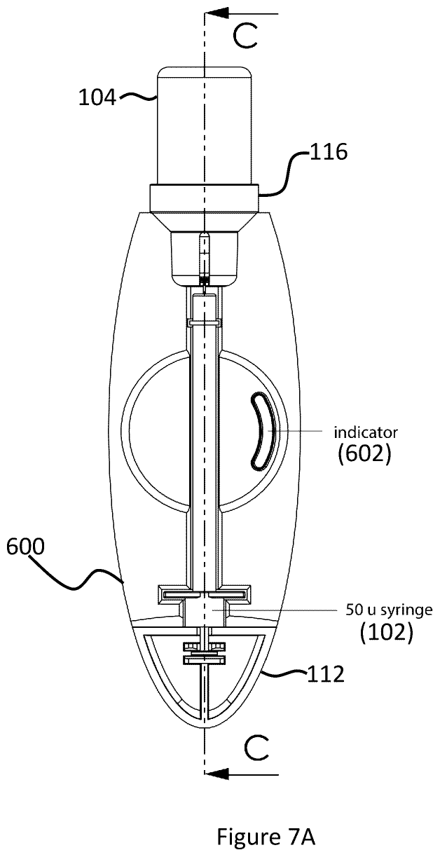

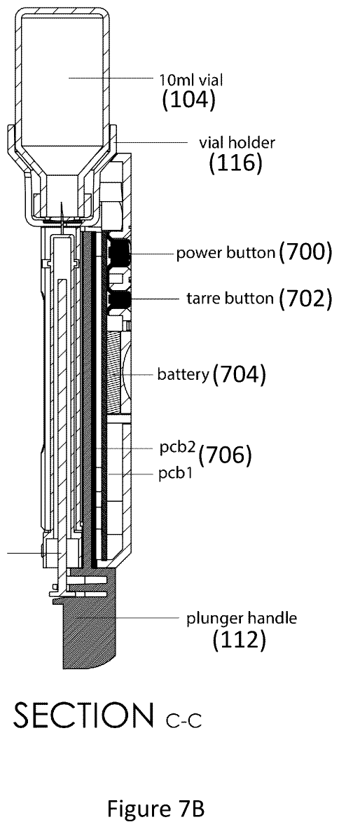

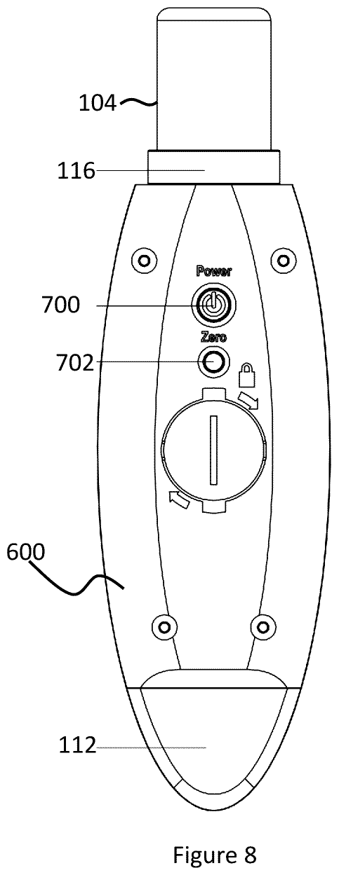

Another embodiment of the present disclosure is depicted in FIGS. 6-8. This embodiment depicts a standalone syringe filling adapter 600 that is not designed to be affixed to a user device 106. Such embodiments may include any combination of the aforementioned features, but relies primarily on visual, tactile, and audible feedback as well as the use of a stop 302 associated with the plunger handle 112 to help the user verify the correct dosage, with embodiments featuring an indicator 602 that may be configured to light or change color upon the user extending the plunger handle 112 to a length indicating a correct dosage or approaching this length. The standalone syringe filling adapter 600 may further comprise a power button 700, tare button 702, battery 704, and Printed Circuit Board (PCB) 706.

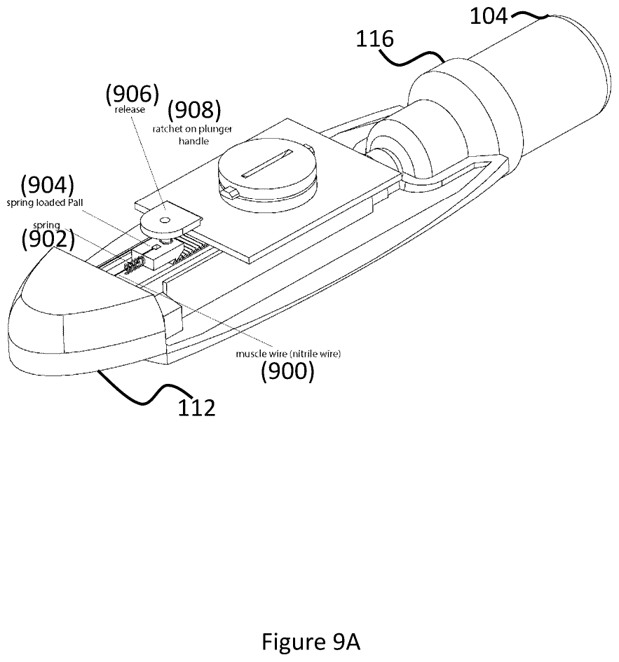

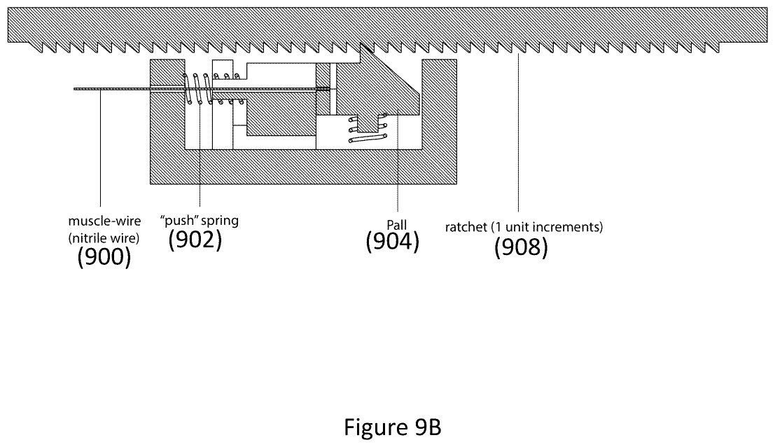

In embodiments, such as that depicted in FIGS. 9A and 9B, a muscle wire plunger handle drive mechanism can be used to extend the plunger handle automatically. Such a mechanism may comprise a spring 902 configured to bias a spring loaded pall 904 in operative communication with a ratchet 908 disposed on the plunger handle 112 towards the vial holder 116, with a muscle wire 900 or shape-memory alloy, such as Nitinol, used to pull the spring loaded pall 904 against spring 902 pressure. Teeth of the ratchet 908 may be spaced apart such that they define 1 unit increments of measure when used with a specific size of syringe 102.

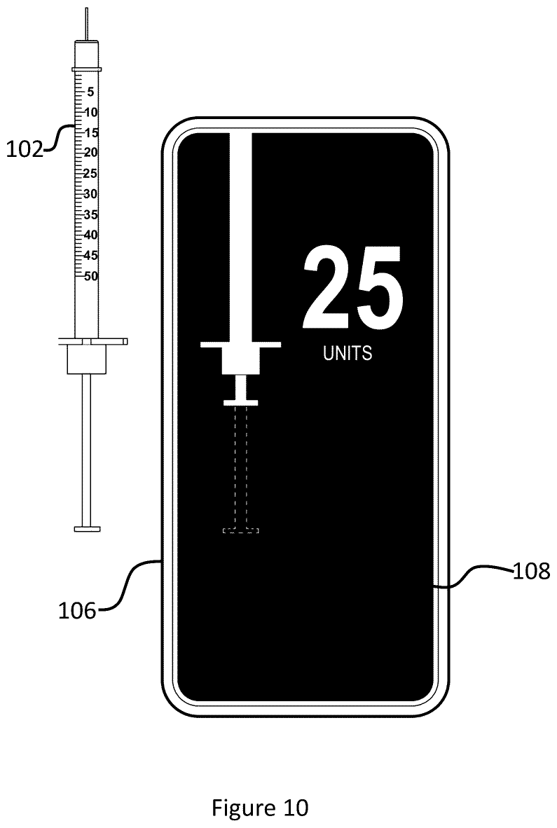

Lastly, embodiments, such as that depicted in FIG. 10, allow use of the user device 106 with standard syringes 102 without a syringe filling adapter 100, standalone or otherwise. In such embodiments, the software running on the user device is configured to show a 1:1 scale graphic depicting a syringe 102; the size of the syringe 102 may be selected by the user or automatically selected by the user device 106, such as by the use of an RFID chip embedded within the syringe 102 that is configured to wirelessly provide this information to the user device 106 when the two are brought into close proximity. The user of such embodiments could fill a standard syringe 102 from a standard vial 104 in the normal way and then compare the filled syringe 102 to the scale graphic to confirm that the syringe 102 was filled correctly.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A system for accurately dosing medication, the system comprising:
a syringe filling adapter configured to hold a syringe and vial such that the syringe is inserted into the vial, the syringe filling adapter further comprising a plunger handle configured for connection to a plunger of the syringe and axial translation, relative to the syringe.

2. The system of claim 1, further comprising a stop associated with the plunger handle and configured to adjustably restrict motion of the plunger handle.

3. The system of claim 1, further comprising a sensor PCB attached to the syringe for capacitive sensing of the plunger position.

4. The system of claim 1, further comprising an actuator linked to the plunger handle, the actuator configured to advance or retract the plunger of the syringe in response to commands from a device.

5. The system of claim 4, wherein the actuator is a muscle-wire actuator.

6. The system of claim 4 wherein the actuator comprises a motor configured to drive a worm-gear.

7. The system of claim 4, wherein the device is configured to provide an input interface for commanding the syringe filling adapter to fill a syringe disposed therein to a target dosage.

8. The system of claim 1, wherein the syringe filling adapter is configured to mount to a device comprising a touch screen display and to hold the syringe adjacent the display and parallel to an edge of the display, and wherein the display is configured to display a target dosage graphic that provides at least one reference point to which the plunger handle should be aligned to fill the syringe to a target dosage.

9. The system of claim 8, wherein the target dosage graphic comprises a numerical value corresponding to the target dosage.

10. The system of claim 8, wherein the target dosage graphic comprises a 1:1 scale graphical representation of the syringe filling adapter with plunger handle extended to a position corresponding to the target dosage, such that the syringe filling adapter completely covers the target dosage graphic when the syringe is filled to the correct dosage.

11. The system of claim 8, wherein the target dosage graphic comprises a graphic to which the plunger handle should abut to fill the syringe to the target dosage.

12. The system of claim 8, wherein the display of the device is a capacitive, touch-sensitive display and wherein the syringe filling adapter includes a capacitive touch member configured to move with the plunger handle, adjacent the display, and thereby to inform the device of a current plunger position.

13. The system of claim 12, wherein the device is configured to obtain an initial plunger handle position through recording the location of the capacitive touch member and then to use an offset from this position to generate the target dosage graphic.

14. The system of claim 12, wherein the device is configured to provide visual, tactile, and/or audible feedback to help users verify the target dosage.

15. The system of claim 14, wherein the feedback is provided in response to the pulling of the plunger handle to the target dosage.

16. The system of claim 14, wherein the feedback is provided as the plunger handle approaches the target dosage.

17. A system for accurately dosing medication, the system comprising:
a display;
at least one processor; and
one or more memories coupled to the at least one processor and having programming that causes the system to perform operations including:
accepting a target dosage from a user;
generating a target dosage graphic that corresponds to a syringe or syringe filling adapter with syringe filled to the target dosage; and
displaying the target dosage graphic on the display.

18. The system for accurately dosing medication of claim 16, wherein the target dosage graphic comprises a graphic to which a plunger handle of the syringe filling adapter should abut to fill a syringe held in the syringe filling adapter to the target dosage.

19. The system for accurately dosing medication of claim 16, wherein the target dosage graphic comprises a 1:1 scale graphic of a syringe filled to the target dosage.

20. A system for accurately dosing medication, the system comprising:
a syringe filling adapter configured to hold a syringe and vial such that the syringe is inserted into the vial, the syringe filling adapter further comprising a plunger handle configured for connection to a plunger of the syringe and axial translation, relative to the syringe;
a stop associated with the plunger handle and configured to adjustably restrict motion of the plunger handle;
wherein the syringe filling adapter is configured to mount to a device comprising a touch screen display and to hold the syringe adjacent the display and parallel to an edge of the display,
wherein the display is configured to display a target dosage graphic that provides at least one reference point to which the plunger handle should be aligned to fill the syringe to a target dosage,
wherein the display of the device is a capacitive, touch-sensitive display,
wherein the syringe filling adapter includes a capacitive touch member configured to move with the plunger handle, adjacent the display, and thereby to inform the device of a current plunger position, and
wherein the device or syringe filling adapter is configured to provide visual, tactile, and/or audible feedback to help users verify the target dosage.
